(19) United States
(12) Patent Application Publication
(10) Pub. No.: US 2023/0184731 A1
(43) Pub. Date: Jun. 15, 2023

(54) METHOD FOR RAPID IN SITU DETECTION OF AMMONIA

(71) Applicant: THE REGENTS OF THE UNIVERSITY OF CALIFORNIA, (US)

(72) Inventors: Dmitry A. FISHMAN, Aliso Viejo, CA (US); Plamen ATANASSOV, Irvine, CA (US); Yuanchao LIU, Irvine, CA (US); Tristan ASSET, Irvine, CA (US); Yechuan CHEN, Irvine, CA (US)

(73) Assignee: THE REGENTS OF THE UNIVERSITY OF CALIFORNIA, Oakland, CA (US)

(21) Appl. No.: 17/923,542

(22) PCT Filed: May 6, 2021
(86) PCT No.: PCT/US2021/031109

§ 371 Date: Nov. 4, 2022

Related U.S. Application Data
(60) Provisional application No. 63/021,912, filed on May 8, 2020.

(51) Int. Cl. G01N 33/00; G01N 21/65

(52) U.S. Cl. CPC G01N 33/0054; G01N 21/658

(57) ABSTRACT

A method for detecting and quantifying an amount of ammonia in a sample by surface-enhanced Raman spectroscopy includes a step of position a liquid or gaseous sample proximate to a detection substrate. Incident light is focused onto the detection substrate while it is positioned proximate to the sample, the incident light having an excitation wavelength from about 500 nm to 800 nm. Raman activity from ammonia proximate to the detection substrate is then detected.

### CROSS-REFERENCE TO RELATED APPLICATIONS

This application claims the benefit of U.S. provisional application Ser. No. 63/021,912 filed May 8, 2020, the disclosure of which is hereby incorporated in its entirety by reference herein.

### TECHNICAL FIELD

In at least one aspect, the present invention is related to methods and devices for measuring the amount of ammonia in a sample.

### BACKGROUND

Ammonia is not only a ubiquitous environmental pollutant for aquatic eutrophication but also an important component of industrial fertilizer currently manufactured via the Haber-Bosch (H-B) process that supports 50% of the nitrogen element in the human body nowadays. Sensitive ammonia detection has been a Holy Grail for many decades as its application is of great importance to fundamental research areas catalysis and electrochemistry¹. Ammonia emission to atmosphere nowadays is fully dominated directly and indirectly by human activity². Thus it is pivotal for areas that are instrumental and influential to our everyday life—cement³, food industry⁴, water quality control⁵, exhaust gas sensing^{6, 7}, chemistry and detection of combustibles⁸, and many more. The literature on the topic is very rich, and strategies to achieve the goal are highly diverse. This makes it virtually impossible to discuss, review and compare all of the approaches. Most notable conventional methods employ electrochemical⁹⁻¹¹ approaches as metal oxides¹²⁻¹⁹, catalytic polymer²⁰⁻²⁷, including novel inkjet printing films²⁸ and graphene/polymer hybrid framework²⁹, and optical techniques³⁰⁻³³, fluorescence-labeled metal-organic-framework³⁴, and nonlinear optics^{35, 36}.

Probably, the most widely accepted optical methods are based on colorimetric approaches and its experimental derivatives³⁷. The popular solution employs Berthelot reaction between ammonia, chlorine, and phenolic compounds, resulting in blue coloration of indophenol dye that can be easily detected by conventional spectrometers³². The following approach allows fine sensitivity down to several tens of parts per billion (ppb)^{32, 33, 38, 39}. Though the method probably champions in overall detection limits, the blue indophenol method requires routine and time-consuming sample handling. It requires an aliquot of 2 mL to be taken out of the reactor each time and mix with other toxic reagents, followed by a 20-60 min aging before the final spectrophotometric analysis. In addition to its intrinsic time and materials consuming nature, colorimetric approaches fail to track the tiny change of on-site ammonia evolution and intermediate species, wherein key factors to reaction mechanism and catalyst design might be lost.

Probing samples directly via the intrinsic absorption or emission properties of ammonia molecule are broadly explored as well. The molecule has several UV⁴⁰ and IR⁴¹⁻⁴³ active bands, allowing non-invasive detection in the liquid or gaseous phase. In UV range, such detection relies on unique laser source and a quite complex optical setup. Moreover, acquisition time is rather long for rapid sample characterization⁴⁰. These techniques proved to be rather sensitive to total ammonia concentrations. However, many concerns have been raised on results being contaminated from ammonia presence in surrounding laboratory and experimental environment. Due to the fact that IR light has long pathway and it is difficult to isolate it from external contamination, labeling with nitrogen isotope has been utilized⁴⁴. Such an approach allows one to identify specific absorption modes associated with N15, similarly to the approach used for nuclear magnetic resonance studies. However, such isotope-related approaches make experiments complex and expensive. Emission techniques, like conventional Raman spectroscopy, have also shown some potential^{35, 36, 45-47}, including UV resonant Raman approach⁴⁸⁻⁵⁴. Though Raman scattering can reveal background free chemical contrast, the signals have shown to be weak and with reasonable experimental conditions it cannot be employed for tracking small concentrations of ammonia.

Many efforts have been made towards the creation of a device to allow rapid ammonia detection in both liquid and gaseous forms. Within the most modern approach, a gas sensor with a detection limit of 10 ppm has been demonstrated using paper with perovskite halide as a sensor material⁵⁵. Another elegant work reports on the fabrication and deployment of a thin film of metal-organic framework (MOF) as a chemical capacitive sensor. MOF is an organic-inorganic crystalline porous material that has unique structural properties compared to conventional porous materials as it allows control of topology and functionality of framework⁵⁶⁻⁵⁸. Detection of level as low as 1 part per million (ppm), with 100 part per billion (ppb) projected limit, has been successfully demonstrated³⁴. Alternative solutions that use metal particles decorated with a graphene-based framework are also of high interest. Such sensors demonstrate a rather short recovery time with a projected detection limit of 45 ppb²⁹.

Although the prior art method works reasonably well, current developments do not allow rapid analysis required for observation of live chemical processes.

Accordingly, there is a need for improved methods and devices for detecting ammonia.

### SUMMARY

In at least one aspect, a method for detecting and quantifying an amount of ammonia in a sample is provided. The method includes steps of providing a liquid or gaseous sample and positioning the sample proximate to a detection substrate. Incident light is focused onto the detection substrate while it is proximate to the sample. Characteristically, the incident light has an excitation wavelength from about 300 nm to 900 nm. Raman activity is detected from ammonia and/ammonia-containing complexes proximate to the detection substrate.

In another aspect, a method for in situ ammonia detection with comparable or superior sensitivity to the complex or known conventional experiments without sample perturbation, alterations, and modifications.

In another aspect, a surface-enhanced Raman spectroscopy (SERS) method to detect on-site ammonia concentration down to single-digit ppm level is provided. It is demonstrated that the method is reversible under different ammonia environments. Moreover, the potential of this method to study the evolution of surface-intermediate species during ENRR and other heterogeneous reactions for ammonia generation is computationally explored. Such low cost, high sensitivity, and feasible method is essential to environmental monitoring and fundamental research on ammonia synthesis and even other nitrogen-related reactions.

In another aspect, a method allowing in situ ammonia detection is provided.

In another aspect, a method allowing revisable ammonia detection is provided.

In another aspect, a method allowing the detection of ammonia in closed environments is provided.

In still another aspect, a device for in situ observation of chemical/catalytic reaction through ammonia release is provided.

In still another aspect, a new elegant yet simple method of sensitive ammonia detection using surface-enhanced Raman scattering (SERS) is provided. This detection method is a non-contact technique that does not rely on the complex chemical design of the sensor or alteration of the sample and solution. The devices implementing this method provide high-speed in-situ detection, with concentrations of 10 ppm concentration of ammonia in water easily observed with a potential 400 ppb projected detection limit. Advantageously, the device is a re-usable closed system, allow detection of both decrease and increase of ammonia concentration without worrying of contamination from the surrounding laboratory environment. Moreover, the described approach is based on very modest experimental conditions, using excitation powers available with most LED light sources (<3 mW) and commercially available plasmonic structures. Since the approach does not rely on complex instrumentation, analysis, or labeling, it is expected to fully democratize ammonia detection, thus drastically boosting the research in many areas of chemistry and surrounding disciplines.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1A. Schematic illustration of a device for holding a sample for which ammonia is to be detected and/or quantified sample by surface-enhanced Raman spectroscopy.

FIG. 1B. Schematic illustration of a device for holding a sample for which ammonia is to be detected and/or quantified sample by surface-enhanced Raman spectroscopy. The device in this figure has a single catalyst bed upstream of the region where Raman activity is measured.

FIG. 1C. Schematic illustration of a device for holding a sample for which ammonia is to be detected and/or quantified sample by surface-enhanced Raman spectroscopy. The device in this figure has two catalyst beds upstream of the region where Raman activity is measured.

FIG. 1D. Schematic illustration of a device for holding a sample for which ammonia is to be detected and/or quantified sample by surface-enhanced Raman spectroscopy. The device in this figure has three catalyst beds upstream of the region where Raman activity is measured.

FIGS. 2A and 2B. Raman spectrum of ammonia in water solution at OH-stretching spectral region. For experiments, 10% (100000 ppm) solution was used. A) The spectrum is represented by multiple Raman active bands from ammonia monomer (NH₃), dimer (NH₃-NH₃), and ammonium (NH₃-H₂O) complexes. B) Depiction of the dimer (NH₃-NH₃) and ammonium (NH₃-H₂O) complexes.

FIGS. 3A and 3B. Schematic representation of SERAS method. Raman experiments are performed in epi geometry using a water immersion objective. Ammonia solution is confined under a mica microscope slide and closed to the laboratory environment.

FIGS. 4A and 4B. SERS spectrum of 50 ppm ammonia solution in water.

FIGS. 5A, 5B, 5C, and 5D. (A) 3260 cm−1 NH₃ line intensity measured at a different place on the substrate surface. The enhancement factor of the substrate does not change drastically across the substrate. (B) Measurements of 100 ppm ammonia and then Millipore water on the same substrate subsequently. Results demonstrate the possibility of measurement of ammonia concentration-time dynamic on a single substrate. (C) Concentration dependence with a linear fit. (D) Measurement of 10 ppm ammonia solution with long acquisition time.

FIGS. 6A and 6B. Concept of ammonia detection simple substrate based on colloidal silver paste.

FIG. 7. Average ammonia detection limit using conventional colloidal silver paste.

FIG. 8. Average ammonia detection limit using colloidal silver ink.

FIGS. 9A, 9B, and 9C. 2D drawings of SERAS device.

FIG. 10. SERAS flow device for ultra-sensitive in-situ ammonia detection. The sample chamber is well sealed on top with a 130-170 um glass cover. The SERES is mounted on a threaded cylindrical holder that is able to seal the chamber bottom and meanwhile adjust the height of SERS to optimize the working distance of Raman objective.

### DETAILED DESCRIPTION

Reference will now be made in detail to presently preferred compositions, embodiments, and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only or both A and B”. In the case of “only A”, the term also covers the possibility that B is absent, i.e. “only A, but not. B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

Ammonia in this context generally means molecular ammonia in the chemical format of NH₃, ammonium cations in a chemical format of NH₄^{+,} and other ammonia-containing complexes such as ammonia dimer, ammonia water complex, etc. It also means any ammonia-related molecule or complex with intrinsic intermolecular and intramolecular vibration Raman signals characteristic of ammonia.

Abbreviations:

“PEEK” means polyether ether ketone.

“PTFE” means polytetrafluoroethylene.

“SERS” or “SERAS” means surface-enhanced Raman spectroscopy.

In an embodiment, a method for detecting and quantifying an amount of ammonia in a sample by surface-enhanced Raman spectroscopy is provided. The method includes a step of providing a liquid or gaseous sample and then positioning the sample proximate to (e.g., within 10 nm to the substrate) to a detection substrate. In a refinement, the sample is at a distance of 0 to 10 nm from the substrate. In another refinement, the sample contacts the substrate. Incident light is focused on the detection substrate while it is proximate to the sample. The incident light has an excitation wavelength from about 300 nm to 900 nm (e.g., 532 nm). As set forth below, the incident light is focused on the sample with a single lens or lens system. The single lens or lens system can be a Raman objective (e.g., a water immersion objective) that also collects Raman scattered light. Raman activity from ammonia proximate to the detection substrate is then detected.

In one variation, Raman activity from ammonia is detected at 700 wavenumbers to 2000 wavenumbers spectral region. In another refinement, Raman activity from ammonia is detected at 2800 wavenumbers to 4000 wavenumbers spectral region. In a refinement, Raman activity can be detected in both these spectral regions for detecting and quantifying the amount of ammonia.

Characteristically, the detection substrate can have a structured surface that includes a structure size that is comparable to or smaller than a wavelength of excitation. In a refinement, the structured surface has nano-sized structures having dimensions less than 500 nm. In a further refinement, the structured surface has nano-sized structures having dimensions from 50 nm to 200 nm. In a refinement, nano-structured means that the structure has at least one dimension less than 500 nm or within the range 50 nm to 200 nm. In one example, the structured surface includes a plurality of pillars. In a refinement, the pillars are composed of silicon or another semiconductor or a dielectric or metal. Typically, the detection substrate includes a base substrate and a metal layer disposed over the base substrate such that the metal layer (e.g., a silver-containing layer) is proximate to the sample. The metal layer is a signal enhancement “hot-spot.” The base substrate can be virtually, any material such as dielectrics, semiconductor, and metals and nanostructures thereof.

The methods set forth herein can detect and quantify the amount of ammonia as a sample. For example, the concentration of ammonia or an ammonia-containing complex can be determined by measuring the amount of Raman scattered light at a wavelength or range of wavelengths that correspond to vibrational modes of ammonia or the ammonia-containing complex. In particular, a calibration curve can be determined from calibration samples of known ammonia or ammonia-containing complex concentrations. Such a calibration curve allows the determination of the concentration of liquid or gaseous samples of unknown concentration by extrapolation or interpolation as needed.

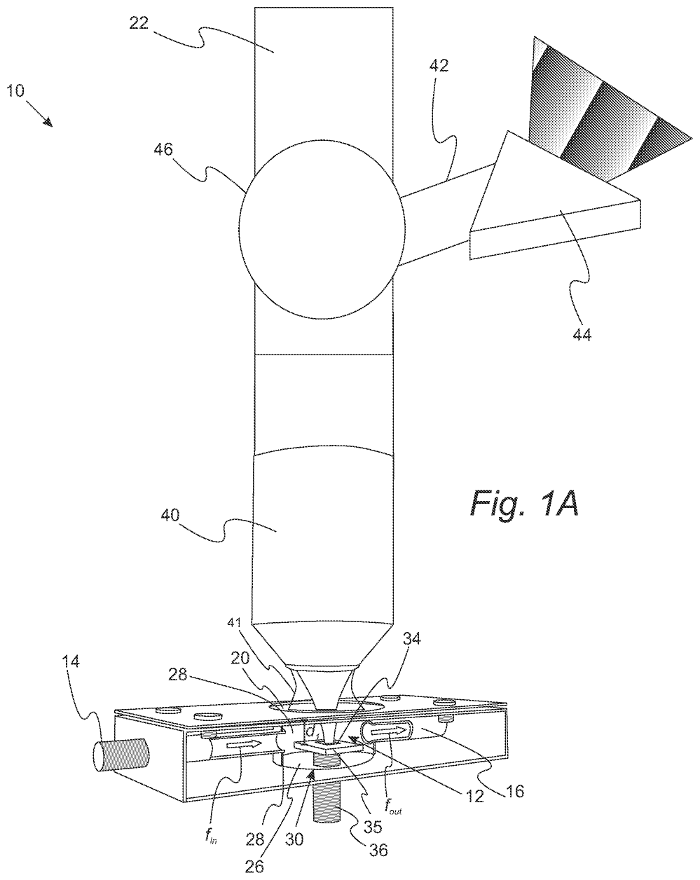

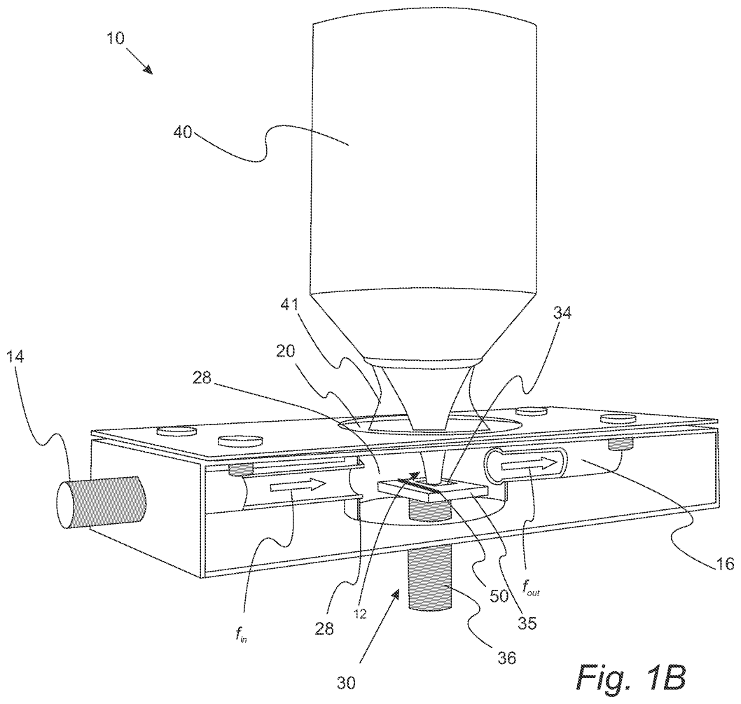

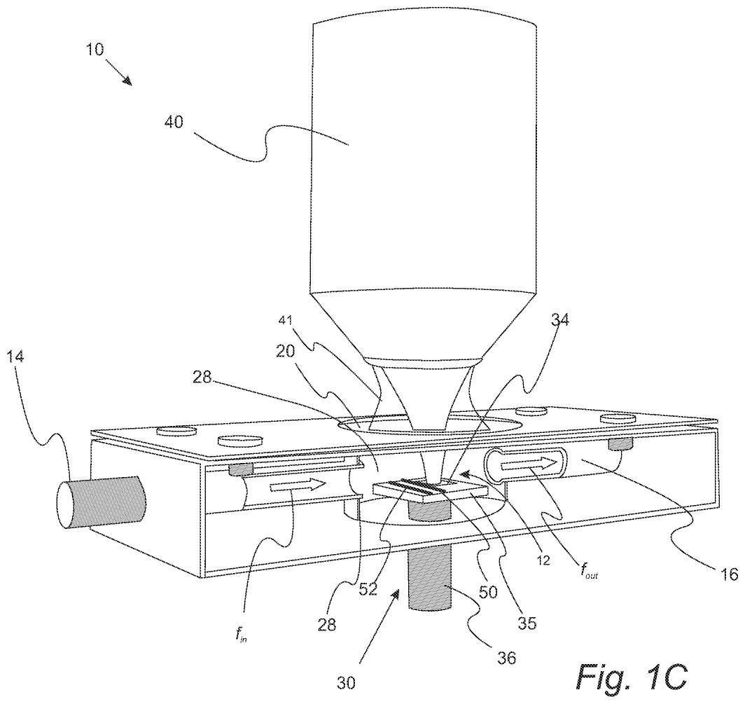

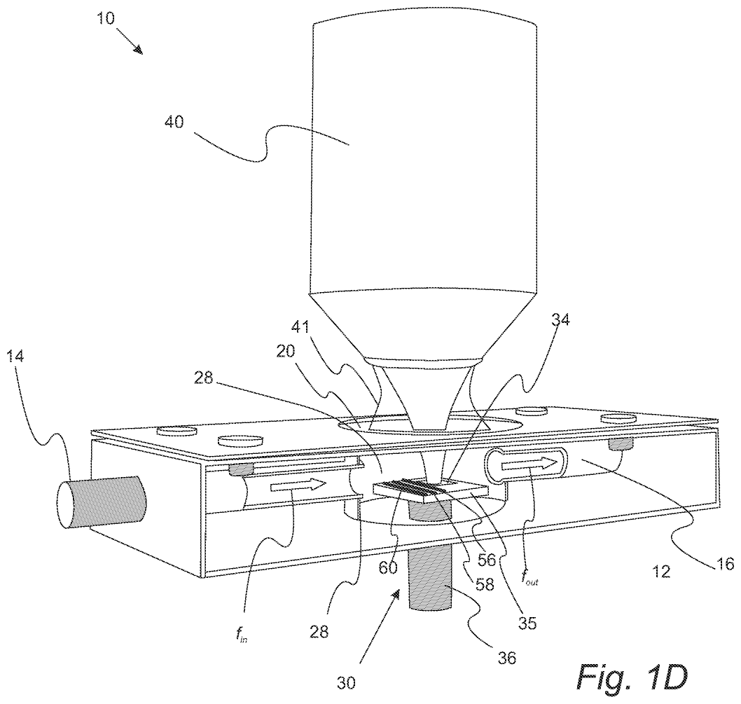

FIGS. 1A, 1B, 1C, and 1D provide schematics of devices for holding a sample for which ammonia is to be detected and/or quantified by the methods set forth herein. Device 10 includes a closed chamber 12 having an inlet port 14 through with a sample flows into closed chamber 12 along flow direction fᵢₙ and an outlet port 16 through which a sample flows out of closed chamber 12 along flow direction fₒᵤₜ. The sample can be either a liquid sample or a gaseous sample. Close chamber 12 includes a top transparent window 20 for passing incident light 22 into the closed chamber 12. Close chamber 12 also includes a bottom wall 26 and sidewall(s) 28. A substrate holder 30 is configured to hold a detection substrate 34 below the top transparent window 20 at a distance d₁. In a refinement, substrate holder 30 includes a stage 35 and a translation component 36 configured to change distance d₁ (e.g., from 1 to 5 mm) and thereby provide distance control. Device 10 is configured for the placement of a single lens or lens system 40 proximate to top transparent window 20. Single lens or lens system 40 focuses the incident light 22 onto detection substrate 34. In a refinement, the single lens or lens system 40 is a Raman objective. In a refinement, the lens or lens system 40 is a water immersion objective that can be immersed is aqueous solution or medium 41. Typically, Raman scattered light 42 from the sample is collected by the single lens or lens system 40 and directed to spectrophotometer 44 by optical component 46 (e.g., a dichroic filter). Detection substrate 34 can have a structured surface having a structure size that is comparable to or smaller than a wavelength of excitation. The details for detection substrate 34 are set forth above.

In some variations, device 10 is operated in a static mode in which the sample is introduced into the closed chamber for at static measurement and then withdrawn after measurement. In other variations, device 10 is operated in a flow mode in which the sample flows through the closed chamber while Raman activity is measured.

Referring to FIGS. 1B, 1C, and 1D, schematics of devices for holding a sample for which ammonia is to be detected and/or quantified sample by surface-enhanced Raman spectroscopy are provided. Each of the devices of these figures includes catalyst beds that allow chemical reactions that generate ammonia to be studied. These variations are advantageously operated in the flow mode, but can still operate in a static mode. FIG. 1B includes a single catalyst bed 50 positioned upstream (with respect to flow direction fᵢₙ) of the region being interrogated for Raman activity. FIG. 1C shows a device that includes two catalyst beds 52 and 54 positioned upstream (with respect to flow direction fᵢₙ) of the region being interrogated for Raman activity. FIG. 1C shows a device that includes three catalyst beds 56, 58, and 60 positioned upstream (with respect to flow direction fᵢₙ) of the region being interrogated for Raman activity. In each of these cases, the catalyst bed may catalyze a reaction that generates ammonia and/or reaction intermediates (e.g., hydrazine in the form of N₂H₄) which then flows over detection substrate 34 to be detected and/or quantified by surface-enhanced Raman spectroscopy.

The following examples illustrate the various embodiments of the present invention. Those skilled in the art will recognize many variations that are within the spirit of the present invention and scope of the claims.

### Observation of Raman activity of NH₃ in NH₃-H₂O

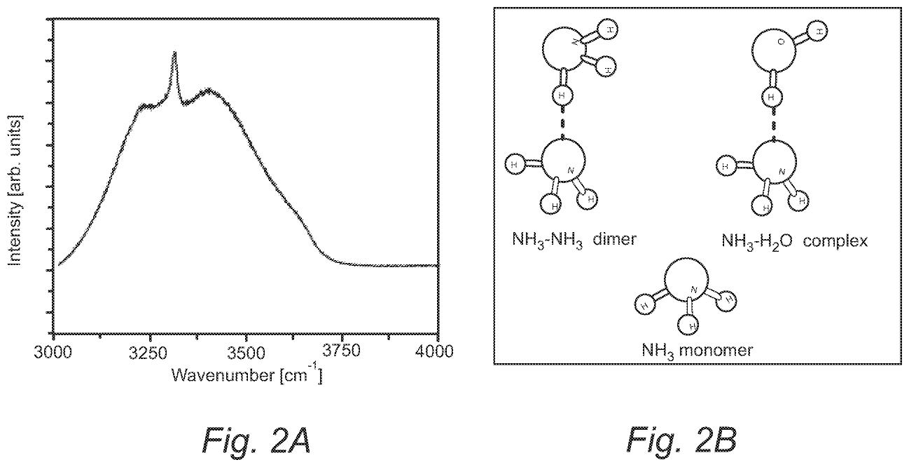

Raman spectra of ammonia solution in water is presented in FIGS. 2A and 2B. In aqueous solution, as expected, the signals of intramolecular OH-stretching modes of water are dominant⁵⁹. This very complex spectral region for liquid water and has been modeled with five closely located bands⁶⁰. Raman activities of ammonia-related structures are also quite present in this region. This region contains many modes of ammonium, i.e. NH₃-H₂O complexes, rotational mode of ammonia monomer (NH₃) and vibration of the dimer (NH₃-NH₃)^{35, 36, 45, 47, 61-64}. Presented data is in good agreement with previous experimental and theoretical studies^{47, 61, 63-65}.

There are several distinctive features in the broad region from 3000 cm⁻¹ to 3500 cm⁻¹. The main contributors are a rather sharp line around 3300 cm⁻¹ and low and high-frequency sidebands to it (FIG. 2). In early works, these region has been assigned to a symmetric vibrational stretch of NH₃ monomer, while observation of triplet line has been explained through rotational mode coupling⁶¹ The origin of strong line around 3300 cm⁻¹ have been experimentally associated with NH₃ mode through analysis of data on molecule confined in silicate melt^{62, 66}. More recent work also has shown that the main peak around 3300 cm⁻¹ has been associated with NH₃ vibration mode with the complementing combination modes⁴⁷. The detailed optical polarization studies in combination with sum frequency generation spectroscopy allowed authors to discuss the origin of sidebands around 3200 cm⁻¹ and 3400 cm⁻¹. Eliminating the possibility of rotational or bend overtone origin, the lines have been assigned to hydrogen bond between ammonia and water molecule in ammonium complex.

Using GF matrix approach, Yeo et al. demonstrated the complexity of spectral information in this region with spectral overlap of all discussed above contributions—ammonia monomer (˜3300 cm⁻¹) and dimer (˜3250 cm⁻¹) of ammonia molecule and ammonium complex (˜3400 cm⁻¹)⁶⁴. Similar calculation accompanied with experimental studies has been done by Ujike et al.³⁶ The calculated modes appear too close for a given line broadening and experimental resolution, thus only comparison of spectral mass shift for signals from liquid ammonia and water solution leads to some conclusions on lines origins. In spectral regions associated normally with bending modes, so-called, fingerprint region, NH₃ and NH₃-H₂O contributions vary drastically and can be more easily distinguished. However, signals strength appears to be three orders of magnitude smaller, preventing the use of these spectral lines for fine ammonia detection. Please, see experimental observation chapter for further discussions on fingerprint region.

### Method: Surface-Enhanced Raman Spectroscopy for Detection of Low Ammonia Concentration

As of now, surface-enhanced Raman spectroscopy (SERS) is a well-used and broadly accepted technique for fine detection down to a single molecule limit. Several mechanisms have been proposed, including electromagnetic⁶⁷ and charge transfer effects⁶⁸. The broadly accepted electromagnetic theory for enhancement relies on excitation of localized surface plasmons—confined surface plasmon polariton when the structure size is comparable or smaller than the wavelength of excitation. The ability of a molecule to come in close proximity to LSP, the so-called “hot spot,” is crucial for the enhancement factor.

Surprisingly, SERS has not been a method of choice for ammonia detection since it has been demonstrated in 1984 in work by Sanchez et al.⁶⁹. Studies on the detection on ammonium nitride reveal great detection limits in fingerprint region⁷⁰. This work used a rather simple experimental approach based on conventional Au-coated Klarite™ substrates⁷¹. However, all the observed lines are attributed to the symmetric stretch of NO₃, and no NH₃ related signals were detected or discussed.

### Experimental Details: Practicing the Invention

Raman spectroscopy

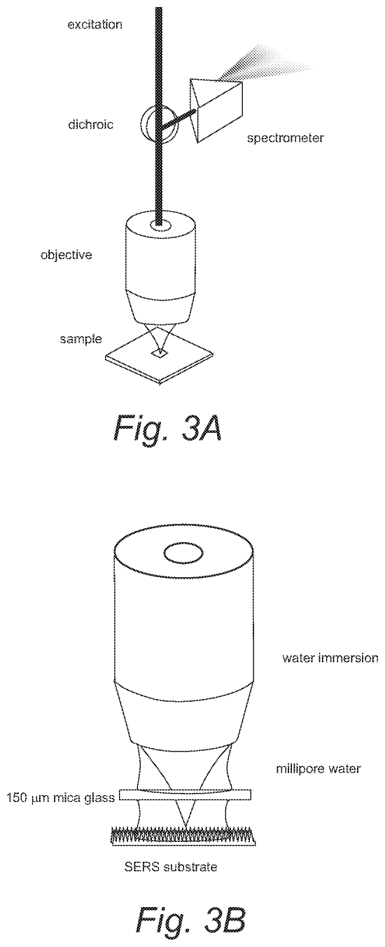

All Raman experiments have been done in conventional epi-geometry and schematically represented in FIGS. 3A and 3B. The experiments have been done using a setup based on the Renishaw InVia Raman microscopy system. For best signal observations, 532 nm has been used as excitation source, with power within 1.5 mW-3 mW at the focal plane. The beam waist in focus is approximated to be ˜500 nm. Raman signal has been collected in epi-geometry using 60×water immersion objective (NA=1.2). Through available other excitation wavelengths (405 nm, 785 nm) the largest signals have been observed only with 532 nm for all used SERS substrates, objective lens and immersion strategies.

SERS Substrate

For experiments with conventional substrates, we used commercially available SERS substrate (SERStrate, Silmeco). The substrates comprise Si-nanopillars coated with the metal of choice. In current work, all detection of ammonia has been demonstrated with Ag coated Si-nanopillars. Similar specification, but Au-coated substrate did not demonstrate any ammonia sensitivity. The following is probably associated with the different capability of ammonia to be attracted to the hot spots of different metals. Prior to experiments, the drop of Millipore water has been deposited on the substrate surface to lean the Si structures in order to increase the interaction area⁷².

For simple homemade substrate, a simple colloidal silver paste and colloidal Ag ink (Sigma Aldrich) deposited on mica coverslip has been used.

Sample Preparation

Ammonia chloride power and ammonia water were used to make various concentrations of ammonium and ammonia solutions, respectively. Due to the different molecular weight of NH₄Cl (NH₃·HCl) and NH₃·H₂O, the concentration of ammonia (in ppm unit) refers to the quantity of NH₃ species as compared to the water background. In this way, for dilute ammonia solution, the molar concentration of NH₄Cl and NH₃·H₂O solutions are consistent.

### Results: Reducing the Invention to Practice

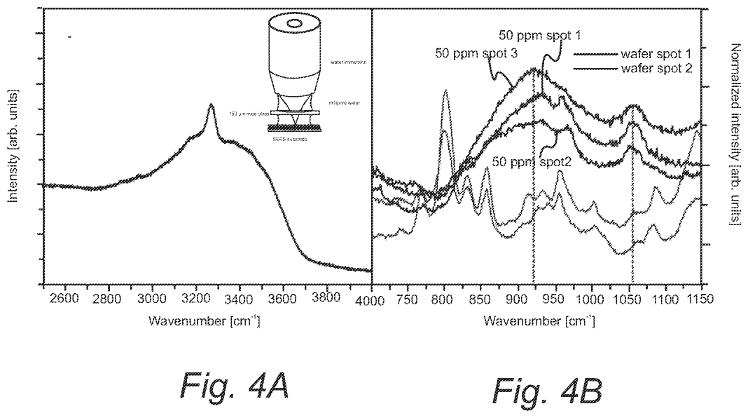

Typical SERS spectra of ammonia solution in water (50 ppm ammonia solution in water) are shown in FIGS. 4A and 4B. The distinctive peak at 3260 cm⁻¹ corresponds to the previously discussed NH₃ dimer signal (FIG. 4A). We believe the slight spectral shift of the peak can be associated with multiple factors. First, the contribution from water, which usually is dominant in this region, is much more suppressed. Second, different species presented in solution (monomer, dimer and ammonium) may have different ability to be confined near hot spot. Per the previous discussion, the following will result in different intensity distributions for sidebands. The following may result in shift of the center of mass for several spectral components in the convolved resultant spectrum. Regardless, the observed signal for 50 ppm solution is comparable to the previously discussed one using conventional Raman experiments for 100,000 ppm concentrated solution in FIG. 2. The following corresponds to an enhancement factor of 2×10³. Such large enhancement makes it possible to demonstrate features at fingerprint range, where ammonia bending modes are expected to be well-separated from each other, and OH-stretch contribution is absent. FIG. 4B demonstrates characteristic response in this spectral range, though some uncertainty due to contamination and enhancement factor deviation across the substrate may alter quantitative data analysis. Qualitatively, line is observed at around 1050 cm⁻¹, which well agrees with previously reported NH₃ symmetrical bending⁶⁴ and as well as NH₃-NH₃ dimer modes³⁶. The origin of broad feature around 900 cm⁻¹ is unknown.

Substrate demonstrated rather homogeneous enhancement throughout its surface with signal variation not to exceed a factor of 2 (FIG. 4A). Important to notice, the detection of ammonia using Ag-coated leaned silicon nanopillars appears to be reversible. To demonstrate the following, first, Si nanopillars were leant, depositing Millipore water droplet on the surface and letting it dry naturally. Raman spectrum of 100 ppm ammonia solution has been measured then, also allowing the sample to dry naturally. Last, Millipore water has been measured, revealing no evidence of ammonia presence. This observation confirms that ammonia does not trap in nanostructure pockets or bound to the metal surface, making the device re-usable and allow detection of ammonia concentration-time dynamics.

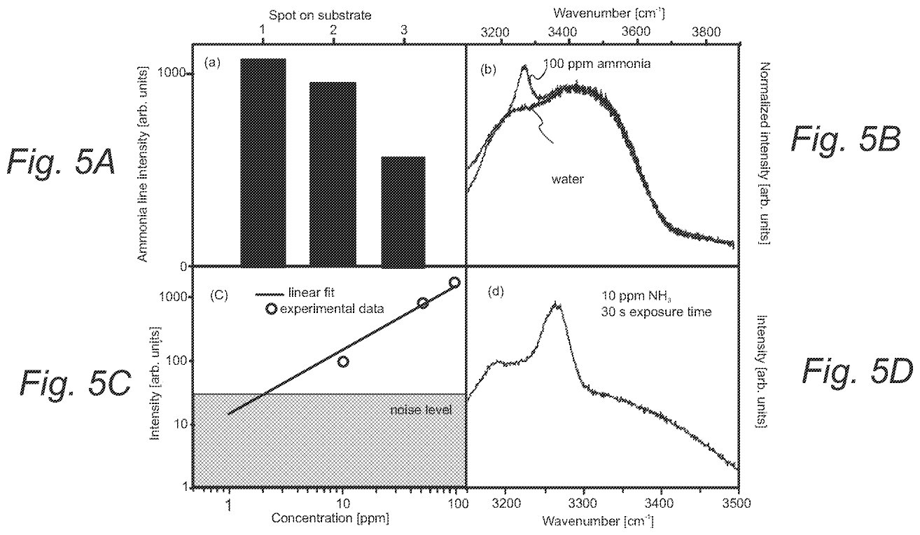

FIG. 5A provides the 3260 cm⁻¹ NH₃ line intensity measured at a different place on the substrate surface. The enhancement factor of the substrate does not change drastically across the substrate. FIG. 5B provides measurements of 100 ppm ammonia and then Millipore water on the same substrate subsequently. Results demonstrate the possibility of measurement of ammonia concentration-time dynamic on a single substrate. FIG. 5C gives the concentration dependence with linear fit. FIG. 5D provides the measurement of 10 ppm ammonia solution with a long acquisition time. The concentration studies summarized in FIG. 5C clearly show linear dependence over the broad range of solutions. The following confirms a previous study (FIG. 5B) that ammonia does not bind to a metal surface and saturation of the detection area does not occur. For used experimental conditions with an acquisition time of 1 s, the detection limit of single-digit ppm can be easily achieved. With an increase of acquisition time, the detected signal quality drastically improves, allowing to project detection limit to 400 ppb of ammonia concentration in label-free close environment experiment. This projected detection limitation already meets the 200 ppb (parts per billion) level that was mostly reported with the indophenol method in the field of ammonia synthesis.

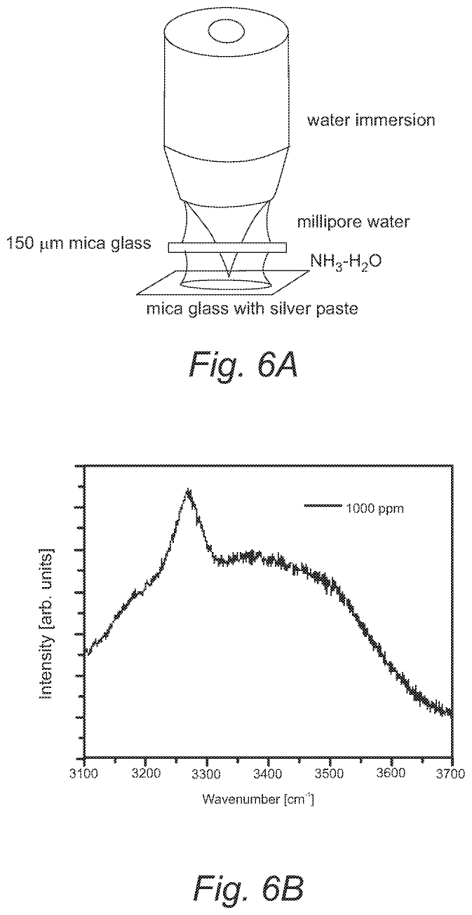

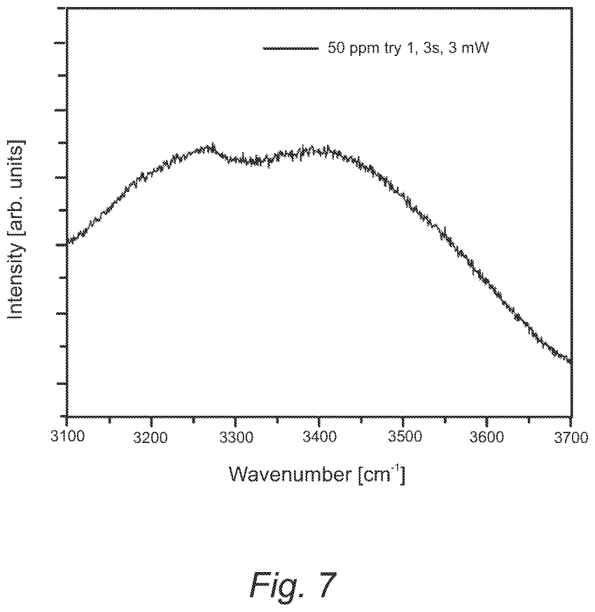

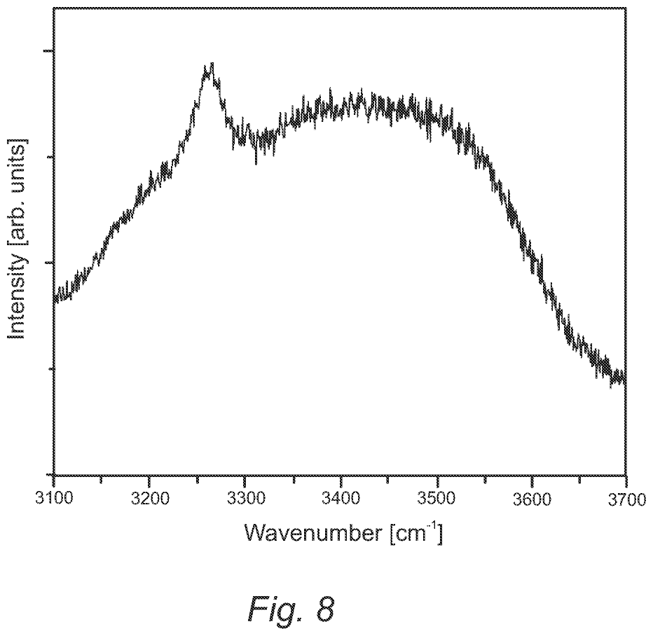

Additional experiments demonstrated that there is no need to employ a specifically designed substrate with complex geometry, rather having uniform Ag coverage of the inert surface with a roughness of the order of 100 nm-200 nm. For such demonstration, commercially available silver paste (Sigma Aldrich) has been deposited on microscopy mica glass coverslip. The resultant cheap substrate demonstrated clear detection of 1000 ppm ammonia concentration (FIG. 6A and 6B). We clearly observed signals for a 50 ppm solution with a projected ultimate limit of detection of 10 ppm (FIG. 7). The following result is an order of magnitude larger than for commercially available substrates. However, the following is believed due to structure dimensions, the content of silver paste, and, most importantly, the presence of oxidation that drastically influenced the effect. Experiments using colloidal silver ink (Sigma Aldrich) gave comparable results to Si-nanopillars SERS substrate (FIG. 8).

Technical Description of Device Design

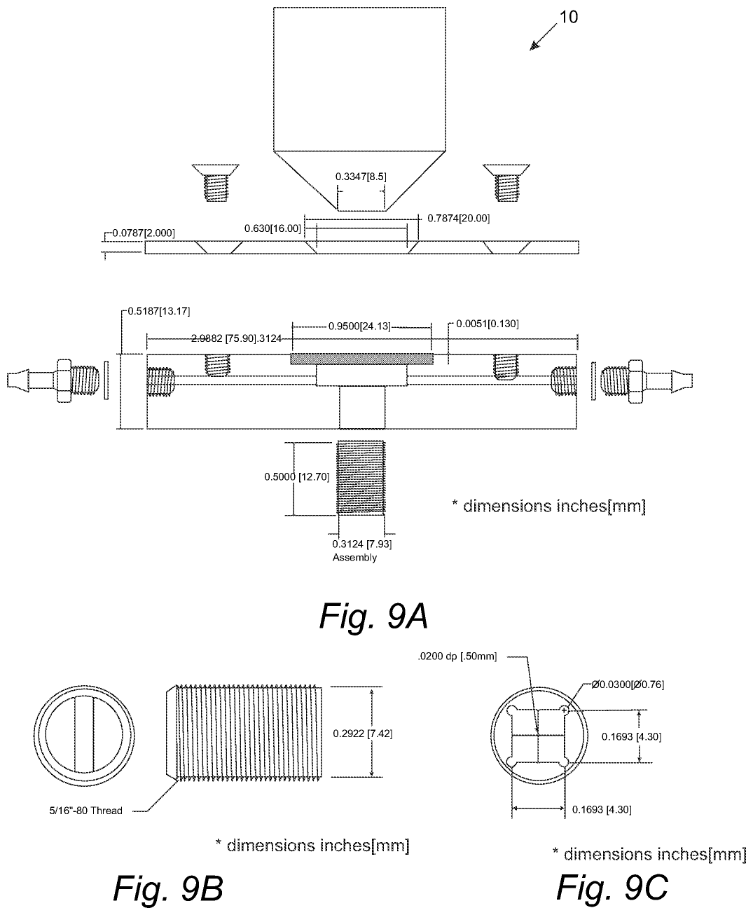

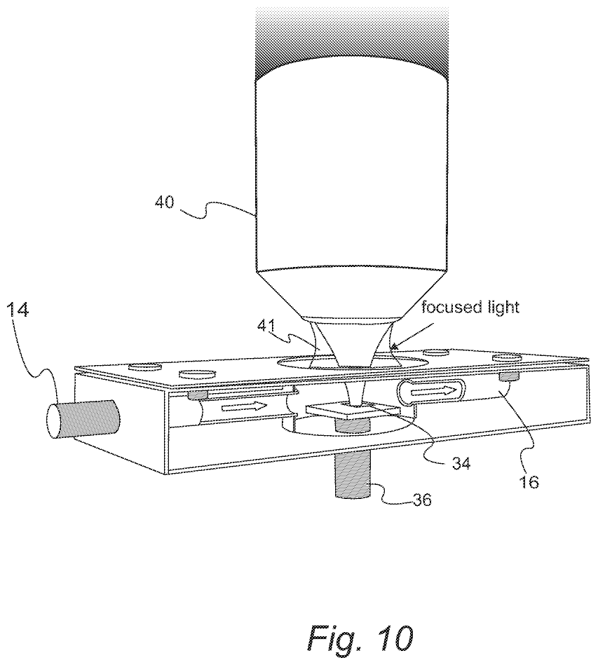

In order to strictly demonstrate the sensitivity, reversibility, and in-situ nature of the methods set forth herein, a flow cell as depicted in FIGS. 9 and 10 was designed with minimum exogenous ammonia contamination and optimum working distance for Raman objective. This design is basically the design set forth in FIGS. 1A-1D described above. As shown in FIGS. 9 and 10, a closed chamber was created by placing a 130-170 μm micro cover glass sandwiched between a PEEK cell cover (with window) and a PTFE gasket. Four bolts applied sufficient pressure on the cell cover to press the glass cover and gasket on the cell base, resulting in a good sealing condition. The utilization of such micro-glass cover allows enough room to adjust the vertical positions of SERS and the Raman objective to achieve the best working distance at 2-2.5 mm. The threaded PEEK holder is able to adjust the height of the SERS while sealing the whole chamber. Depicted in FIG. 10 is single lens or lens system 40, which is immersed in immersion water 41. Sinble lens or lens system 40 focuses light onto sample 34.

In a typical analysis, Millipore pore water is flowed through the whole cell, and 5*5 data points are collected at different spots on SERS, which allows to fully evaluate the water background. The water enters through inlet 14 and exits through outlet 16. Then, certain concentrations of ammonia solutions (pH=pKa, pKa±2) are flow through the cell for 10 min to replace the water, and then analysis with different Raman parameters is conducted. Finally, the chamber is washed with Millipore water again to evaluate the reversibility of the whole process.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

References:
1. Andersen, S. Z. et al. A rigorous electrochemical ammonia synthesis protocol with quantitative isotope measurements. Nature 570, 504-508 (2019).
2. Warneck, P. Chemistry of the natural atmosphere. (Elsevier, 1999).
3. Cheney, J. L. & Knapp, K. T. A Study of Ammonia Source at a Portland Cement Production Plant. JAPCA 37, 1298-1302 (1987).
4. Ampuero, S. & Bosset, J O The electronic nose applied to dairy products: a review. Sensors and Actuators B: Chemical 94, 1-12 (2003).
5. Association, APH, Association, AWW, Federation, WPC & Federation, W.E. Standard Methods for the Examination of Water and Wastewater. (American Public Health Association., 1995).
6. Docquier, N. & Candel, S. Combustion control and sensors: a review. Progress in Energy and Combustion Science 28, 107-150 (2002).
7. Riegel, J., Neumann, H. & Wiedenmann, H. M. Exhaust gas sensors for automotive emission control. Solid State Ionics 152-153, 783-800 (2002).
8. Kohl, D. Function and applications of gas sensors. Journal of Physics D: Applied Physics 34, R125-R149 (2001).
9. Chang, S. C., Stetter, J. R. & Cha, C. S. Amperometric gas sensors. Talanta 40, 461-477 (1993).
10. Lundström, I., Svensson, C., Spetz, A., Sundgren, H. & Winquist, F. From hydrogen sensors to olfactory images-twenty years with catalytic field-effect devices. Sensors and Actuators: B. Chemical 13, 16-23 (1993).
11. Timmer, B., Olthuis, W. & Berg, A. v. d. Ammonia sensors and their applications—a review. Sensors and Actuators B: Chemical 107, 666-677 (2005).
12. Clifford, P. K. & Tuma, D. T. Characteristics of semiconductor gas sensors I. Steady state gas response. Sensors and Actuators 3, 233-254 (1982).
13. Hübner, H. P. & Drost, S. Tin oxide gas sensors: An analytical comparison of gas-sensitive and non-gas-sensitive thin films. Sensors and Actuators B: Chemical 4, 463-466 (1991).
14. Imawan, C., Solzbacher, F., Steffes, H. & Obermeier, E. Gas-sensing characteristics of modified-MoO3 thin films using Ti-overlayers for NH3 gas sensors. Sensors and Actuators B: Chemical 64, 193-197 (2000).
15. Srivastava, R. K., Lal, P., Dwivedi, R. & Srivastava, S. K. Sensing mechanism in tin oxide-based thick-film gas sensors. Sensors and Actuators B: Chemical 21, 213-218 (1994).
16. Wang, X., Miura, N. & Yamazoe, N. Study of WO3-based sensing materials for NH3 and NO detection. Sensors and Actuators B: Chemical 66, 74-76 (2000).
17. Xu, C., Miura, N., Ishida, Y., Matsuda, K. & Yamazoe, N. Selective detection of NH3 over NO in combustion exhausts by using Au and MoO3 doubly promoted WO3 element. Sensors and Actuators B: Chemical 65, 163-165 (2000).
18. Yamazoe, N. New approaches for improving semiconductor gas sensors. Sensors and Actuators B: Chemical 5, 7-19 (1991).
19.Zakrzewska, K. Mixed oxides as gas sensors. Thin Solid Films 391, 229-238 (2001).
20.Cai, Q. Y., Jain, M. K. & Grimes, C. A. A wireless, remote query ammonia sensor. Sensors and Actuators B: Chemical 77, 614-619 (2001).
21. Chabukswar, V. V., Pethkar, S. & Athawale, A. A. Acrylic acid doped polyaniline as an ammonia sensor. Sensors and Actuators B: Chemical 77, 657-663 (2001).
22. Heiduschka, P., Preschel, M., Rösch, M. & Göpel, W. Regeneration of an electropolymerised polypyrrole layer for the amperometric detection of ammonia. Biosensors and Bioelectronics 12, 1227-1231 (1997).
23. Kukla, A. L., Shirshov, Y. M. & Piletsky, S. A. Ammonia sensors based on sensitive polyaniline films. Sensors and Actuators B: Chemical 37, 135-140 (1996).
24. Lähdesmäki, I., Kubiak, W. W., Lewenstam, A. & Ivaska, A. Interferences in a polypyrrole-based amperometric ammonia sensor. Talanta 52, 269-275 (2000).
25. Lähdesmäki, I., Lewenstam, A. & Ivaska, A. A polypyrrole-based amperometric ammonia sensor. Talanta 43, 125-134 (1996).
26. Nicolas-Debarnot, D. & Poncin-Epaillard, F. Polyaniline as a new sensitive layer for gas sensors. Analytica Chimica Acta 475, 1-15 (2003).
27. Palmqvist, E., Berggren Kriz, C., Svanberg, K., Khayyami, M. & Kriz, D. DC-resistometric urea sensitive device utilizing a conducting polymer film for the gas-phase detection of ammonia. Biosensors and Bioelectronics 10, 283-287 (1995).
28. Lv, D. et al. Enhanced flexible room temperature ammonia sensor based on PEDOT: PSS thin film with FeCl3 additives prepared by inkjet printing. Sensors and Actuators B: Chemical 298, 126890 (2019).
29. Ahmadi Tabr, F., Salehiravesh, F., Adelnia, H., Gavgani, J. N. & Mahyari, M. High sensitivity ammonia detection using metal nanoparticles decorated on graphene macroporous frameworks/polyaniline hybrid. Talanta 197, 457-464 (2019).
30. Giovannozzi, A. M. et al. An infrared spectroscopy method to detect ammonia in gastric juice. Analytical and Bioanalytical Chemistry 407, 8423-8431 (2015).
31. Max, J.-J. & Chapados, C. Aqueous ammonia and ammonium chloride hydrates: Principal infrared spectra. Journal of Molecular Structure 1046, 124-135 (2013).
32. Patton, C. J. & Crouch, S. R. Spectrophotometric and kinetics investigation of the Berthelot reaction for the determination of ammonia. Analytical Chemistry 49, 464-469 (1977).
33. Tzollas, N. M., Zachariadis, G. A., Anthemidis, A. N. & Stratis, J. A. A new approach to indophenol blue method for determination of ammonium in geothermal waters with high mineral content. International Journal of Environmental Analytical Chemistry 90, 115-126 (2010).
34. Assen, A H, Yassine, O., Shekhah, O., Eddaoudi, M. & Salama, K. N. MOFs for the Sensitive Detection of Ammonia: Deployment of fcu-MOF Thin Films as Effective Chemical Capacitive Sensors. ACS Sensors 2, 1294-1301 (2017).
35. Li, Y. & Keppler, H. Nitrogen speciation in mantle and crustal fluids. Geochimica et Cosmochimica Acta 129, 13-32 (2014).
36. Ujike, T. & Tominaga, Y. Raman spectral analysis of liquid ammonia and aqueous solution of ammonia. Journal of Raman Spectroscopy 33, 485-493 (2002).
37. Searle, P. L. THE BERTHELOT OR INDOPHENOL REACTION AND ITS USE IN THE ANALYTICAL-CHEMISTRY OF NITROGEN—A REVIEW. Analyst 109, 549-568 (1984).
38. Bolleter, W. T., Bushman, C. J. & Tidwell, P. W. Spectrophotometric Determination of Ammonia as Indophenol. Analytical Chemistry 33, 592-594 (1961).
39. Zhao, Y. et al. Ammonia Detection Methods in Photocatalytic and Electrocatalytic Experiments: How to Improve the Reliability of NH3 Production Rates? Advanced Science 6, 1802109 (2019).
40. Mount, G. H. et al. Measurement of atmospheric ammonia at a dairy using differential optical absorption spectroscopy in the mid-ultraviolet. Atmospheric Environment 36, 1799-1810 (2002).
41. Bromberg, A., Kimel, S. & Ron, A. Infrared spectrum of liquid and crystalline ammonia. Chemical Physics Letters 46, 262-266 (1977).
42. Kindt, J. & Schmuttenmaer, C. Far-infrared absorption spectra of water, ammonia, and chloroform calculated from instantaneous normal mode theory. The Journal of chemical physics 106, 4389-4400 (1997).
43. Kroutil, R., Combs, R., Knapp, R. & Godfrey, J. Infrared interferogram analysis for ammonia detection with passive FTIR spectrometry, Vol. 2763. (SPIE, 1996).
44. McEnaney, J. M. et al. Ammonia synthesis from N2 and H2O using a lithium cycling electrification strategy at atmospheric pressure. Energy & Environmental Science 10, 1621-1630 (2017).
45. Aggarwal, R. L., Farrar, L. W., Cecca, S D. & Jeys, T. H. Raman spectra and cross sections of ammonia, chlorine, hydrogen sulfide, phosgene, and sulfur dioxide toxic gases in the fingerprint region 400-1400 cm−1. AIP Advances 6, 025310 (2016).
46. Gardiner, D., Hester, R. & Grossman, W. Ammonia in the liquid state and in solution: a Raman study. Journal of Raman Spectroscopy 1, 87-95 (1973).
47. Simonelli, D. & Shultz, M. J. Temperature Dependence for the Relative Raman Cross Section of the Ammonia/Water Complex. Journal of Molecular Spectroscopy 205, 221-226 (2001).
48. Ghosh, M., Wang, L. & Asher, S. A. Deep-Ultraviolet Resonance Raman Excitation Profiles of NH4NO3, PETN, TNT, HMX, and RDX. Appl. Spectrosc. 66, 1013-1021 (2012).
49. Tuschel, D. D., Mikhonin, A. V., Lemoff, B. E. & Asher, S. A. Deep Ultraviolet Resonance Raman Excitation Enables Explosives Detection. Appl. Spectrosc. 64, 425-432 (2010).
50. Bykov, S. et al. Deep UV resonance Raman spectroscopy for standoff explosive detection (Conference Presentation), Vol. 11086. (SPIE, 2019).
51. Asher, S. A. UV Resonance Raman Studies of Molecular Structure and Dynamics: Applications in Physical and Biophysical Chemistry. Annual Review of Physical Chemistry 39, 537-588 (1988).
52. Buckley, K. & Ryder, A.G. Applications of Raman Spectroscopy in Biopharmaceutical Manufacturing: A Short Review. Appl. Spectrosc. 71, 1085-1116 (2017).
53. López-López, M. & García-Ruiz, C. Infrared and Raman spectroscopy techniques applied to identification of explosives. TrAC Trends in Analytical Chemistry 54, 36-44 (2014).
54. Gares, K. L., Hufziger, K. T., Bykov, S. V. & Asher, S. A. Review of explosive detection methodologies and the emergence of standoff deep UV resonance Raman. Journal of Raman Spectroscopy 47, 124-141 (2016).
55. Maity, A., Raychaudhuri, A. K. & Ghosh, B. High sensitivity NH3 gas sensor with electrical readout made on paper with perovskite halide as sensor material. Scientific Reports 9, 7777 (2019).
56. Kreno, L. E. et al. Metal-organic framework materials as chemical sensors. Chemical reviews 112, 1105-1125 (2012).
57. Kuppler, R. J. et al. Potential applications of metal-organic frameworks. Coordination Chemistry Reviews 253, 3042-3066 (2009).
58. Xue, D.-X. et al. Tunable rare earth fcu-MOF platform: access to adsorption kinetics driven gas/vapor separations via pore size contraction. Journal of the American Chemical Society 137, 5034-5040 (2015).
59. Cross, P. C., Burnham, J. & Leighton, P. A. The Raman Spectrum and the Structure of Water. Journal of the American Chemical Society 59, 1134-1147 (1937).
60. Carey, D. M. & Korenowski, G. M. Measurement of the Raman spectrum of liquid water. The Journal of Chemical Physics 108, 2669-2675 (1998).
61. Langseth, A. Feinstruktur von Ramanbanden-II. Das Ramanspektrum von Ammoniak in wässeriger Lösung. Zeitschrift für Physik 77, 60-71 (1932).
62. Mysen, B. O. & Fogel, M. L. Nitrogen and hydrogen isotope compositions and solubility in silicate melts in equilibrium with reduced (N+H)-bearing fluids at high pressure and temperature: Effects of melt structure. American Mineralogist 95, 987-999 (2010).
63. Sosa, C. P., Carpenter, J E & Novoa, J. J. in Chemical Applications of Density—Functional Theory, Vol. 629 131-141 (American Chemical Society, 1996).
64. Yeo, G. A. & Ford, T. A. The matrix isolation infrared spectrum of the water-ammonia complex. Spectrochimica Acta Part A: Molecular Spectroscopy 47, 485-492 (1991).
65. Marchand, A. ETUDE DU TRIPLET RAMAN DE LAMMONIAC VERS 3300 CM−1. COMPTES RENDUS HEBDOMADAIRES DES SEANCES DE L ACADEMIE DES SCIENCES 232, 395-397 (1951).
66. Mysen, B. O., Yamashita, S. & Chertkova, N. Amorphous materials: Properties, structure, and durability†: Solubility and solution mechanisms of NOH volatiles in silicate melts at high pressure and temperature—amine groups and hydrogen fugacity. American Mineralogist 93, 1760-1770 (2008).
67. Jeanmaire, D. L. & Van Duyne, R. P. Surface raman spectroelectrochemistry: Part I. Heterocyclic, aromatic, and aliphatic amines adsorbed on the anodized silver electrode. Journal of Electroanalytical Chemistry and Interfacial Electrochemistry 84, 1-20 (1977).
68. Albrecht, M.G. & Creighton, J.A. Anomalously intense Raman spectra of pyridine at a silver electrode. Journal of the American Chemical Society 99, 5215-5217 (1977).
69. Sanchez, L. A., Lombardi, J. R. & Birke, R. L. Surface enhanced Raman scattering of ammonia. Chemical Physics Letters 108, 45-50 (1984).
70. Farrell, M. E., Holthoff, E L & Pellegrino, P. M. Surface-Enhanced Raman Scattering Detection of Ammonium Nitrate Samples Fabricated Using Drop-on-Demand Inkjet Technology. Appl. Spectrosc. 68, 287-296 (2014).
71. Mosier-Boss, P. A. Review of SERS Substrates for Chemical Sensing. Nanomaterials (Basel) 7, 142 (2017).
72. Schmidt, M. S., Hubner, J. & Boisen, A. Large Area Fabrication of Leaning Silicon Nanopillars for Surface Enhanced Raman Spectroscopy. Advanced Materials 24, OP11-0P18 (2012).

What is claimed is:

1. A method for detecting and quantifying an amount of ammonia in a sample by surface-enhanced Raman spectroscopy, the method comprising:
providing a liquid or gaseous sample;
positioning the sample within 10 nm to a detection substrate;
focusing incident light onto the detection substrate while it is within 10 nm from the sample, the incident light having an excitation wavelength from about 300 nm to 900 nm; and
detecting Raman activity from ammonia proximate to the detection substrate.

2. The method of claim 1 wherein Raman activity from ammonia is detected at 700 wavenumbers to 2000 wavenumbers spectral region.

3. The method of claim 1 wherein Raman activity from ammonia is detected at 2800 wavenumbers to 4000 wavenumbers.

4. The method of claim 1 wherein the detection substrate has a structured surface having a structure size that is comparable to or smaller than a wavelength of excitation.

5. The method of claim 4 wherein the structured surface has nano-sized structures having dimensions less than 500 nm.

6. The method of claim 5 wherein the structured surface has nano-sized structures having dimensions from 50 nm to 200 nm.

7. The method of claim 4 wherein the structured surface includes a plurality of pillars.

8. The method of claim 1 wherein the detection substrate includes a base substrate and a metal layer disposed over the base substrate, the metal layer being proximate to the sample.

9. The method of claim 8 wherein the metal layer is a silver-containing layer.

10. The method of claim 1 wherein the excitation wavelength is about 532 nm.

11. The method of claim 1 wherein a concentration of ammonia or an ammonia-containing complex is determined by measuring the amount of Raman scattered light at a wavelength or range of wavelengths that correspond to vibrational modes of ammonia or the ammonia-containing complex.

12. The method of claim 11 wherein a calibration curve is determined from calibration samples of known ammonia or ammonia-containing complex concentrations, the calibration curve allowing determination of the concentration of liquid or gaseous samples of unknown concentration.

13. The method of claim 1 wherein the incident light is focused on the sample with a single lens or lens system that also collects Raman scattered light.

14. The method of claim 13 wherein the single lens or lens system can be immersed into aqueous solution.

15. A device for holding a sample for which ammonia is to be detected and/or quantified sample by surface-enhanced Raman spectroscopy, the device comprising:
a closed chamber including an inlet port through which the sample flows into the closed chamber and an outlet port through which the sample flows out of the closed chamber, the closed chamber also including a top transparent window for passing incident light 22 into the closed chamber, a bottom wall, and a sidewall(s);
a detection substrate; and
a substrate holder configured to hold the detection substrate below the top transparent window at a working distance, the substrate holder including a translation component configured to change the working distance, wherein the device is configured for placement of a single lens or lens system proximate to top transparent window and wherein a single lens or lens system focuses the incident light onto the detection substrate.

16. The device of claim 15 wherein the single lens or lens system can be immersed into an aqueous solution.

17. The device of claim 15 wherein the substrate holder is configured to adjust the working distance to be from 0 to 5 mm.

18. The device of claim 15 wherein the detection substrate has a structured surface having a structure size that is comparable to or smaller than a wavelength of excitation.

19. The device of claim 18 wherein the structured surface has nano-sized structures having dimensions less than 500 nm.

20. The device of claim 19 wherein the structured surface has nano-sized structures having dimensions from 50 nm to 200 nm.

21. The device of claim 19 wherein the structured surface includes a plurality of pillars.

22. The device of claim 15 wherein the detection substrate includes a base substrate and a metal layer disposed over the base substrate, the metal layer being proximate to the sample.

23. The device of claim 22 wherein the metal layer is a silver-containing layer.

24. The device of claim 15 wherein the sample is introduced into the closed chamber for at static measurement.

25. The device of claim 15 wherein the sample flows through the closed chamber while emission is measured.

26. The device of claim 25 further comprising a catalyst bed upstream of a region being interrogated for Raman activity, the catalyst bed catalyzing chemical reactions that generate ammonia and/or reaction intermediates to be detected and/or quantified by surface-enhanced Raman spectroscopy.

27. The device of claim 25 further comprising a plurality of catalyst beds upstream of a region being interrogated for Raman activity, the plurality of catalyst beds catalyzing chemical reactions that generate ammonia to be detected and/or quantified by surface-enhanced Raman spectroscopy.
